(19) United States
(12) Patent Application Publication
(10) Pub. No.: US 2019/0099117 A1
(43) Pub. Date: Apr. 4, 2019

(54) FINGER CUFF HAVING VIBRATION MECHANISM FOR USE IN PERFORMING A FINGER PRICK

(71) Applicant: RELIANT IMMUNE DIAGNOSTICS, INC, Austin, TX (US)

(72) Inventors: JOVAN HUTTON PULITZER, FRISCO, TX (US); HENRY JOSEPH LEGERE, III, AUSTIN, TX (US)

(21) Appl. No.: 16/136,507

(22) Filed: Sep. 20, 2018

Related U.S. Application Data
(60) Provisional application No. 62/566,608, filed on Oct. 2, 2017.

(51) Int. Cl. A61B 5/15

(52) U.S. Cl. CPC A61B 5/150137

(57) ABSTRACT

A vibrating finger cuff for use in performing a finger prick comprises a body having a first end and a second end, wherein the first end having a first opening and the second end having a second opening, and wherein a finger is inserted into the first opening until the finger exits the hollow body at the second opening, a housing secured to an outside surface of the hollow body, the housing including within a vibrator motor, a negative battery contact, a switch contact, a negative motor wire connected between the vibrator motor and the negative battery contact, a positive motor wire connected between the vibrator motor and the switch contact, and a battery housing containing a battery and a positive battery contact, wherein the positive battery contact extends upward from the battery housing so that it contacts the switch contact, and wherein the negative battery contact contacts the battery.

### CROSS-REFERENCE TO RELATED APPLICATIONS

This application claims the benefit of U.S. Provisional Application No. 62/566,608, filed on Oct. 2, 2017, which is incorporated by reference herein in its entirety.

### BACKGROUND

A finger prick procedure to draw blood for use in medical diagnostic tests is common, especially in home diagnostic tests such as blood glucose level tests. Finger pricks can be painful, especially when the same area of the finger is pricked over and over again. Topical or oral anesthetics may not be practical or desired when performing a test. However, the gate theory of pain is a theory that asserts that non-painful input closes the gates to painful input, which prevents pain sensation from traveling to the central nervous system. Thus, stimulation by non-noxious input is able to suppress pain. Therefore, what is needed is an apparatus for providing non-painful input to the finger or nearby areas while a finger prick is performed.

### SUMMARY

In one aspect thereof, a vibrating finger cuff for use in performing a finger prick is provided. The vibrating finger cuff comprises a hollow body having a first end and a second end, wherein the first end having a first opening and the second end having a second opening, and wherein a finger is inserted into the first opening until the finger exits the hollow body at the second opening, a housing secured to an outside surface of the hollow body, the housing including within a vibrator motor, a negative battery contact, a switch contact, a negative motor wire connected between the vibrator motor and the negative battery contact, a positive motor wire connected between the vibrator motor and the switch contact, and a battery housing containing a battery and a positive battery contact, wherein the positive battery contact extends upward from the battery housing so that it contacts the switch contact, and wherein the negative battery contact contacts the battery.

In another aspect thereof, a method for performing a finger prick using a vibration apparatus is provided. The method comprises placing the vibration apparatus at a designated location on a user's body, wherein the vibration apparatus includes a hollow body that can be placed around the designated location and a housing fixedly coupled to the hollow body, the housing including a vibration mechanism and a battery. The method further comprises activating the vibration apparatus to initiate a vibration effect at the designated location, allowing the vibration effect to continue at the designated location for at least a predetermined amount of time, performing a finger prick at one of the user's fingertips upon reaching at least the predetermined amount of time, deactivating the vibration apparatus, and removing the vibration apparatus from the designated location.

### BRIEF DESCRIPTION OF THE DRAWINGS

For a more complete understanding, reference is now made to the following description taken in conjunction with the accompanying Drawings in which:

FIG. 1 illustrates a perspective view of one embodiment of a vibrating finger ring;

FIG. 2 illustrates a top view of a housing a securing member of one embodiment of a vibrating finger ring;

FIG. 3 illustrates an exploded perspective view of internal vibration components of a housing and a securing member of one embodiment of a vibrating finger ring;

FIG. 4 illustrates a perspective view of one embodiment of a vibrating finger ring worn a hand;

FIG. 5 illustrates one embodiment of a vibrating finger sleeve;

FIG. 6 illustrates another embodiment of a vibrating finger sleeve;

FIG. 7 illustrates a perspective view of one embodiment of a vibrating finger sleeve worn a hand;

FIGS. 8A-8D illustrate various locations on the hand or wrist that may be used with the vibration apparatuses and methods described herein;

FIG. 9 illustrates one embodiment of a vibrating band apparatus;

FIG. 10 illustrates one example of a location on a user's body for placing a vibration apparatus;

FIG. 11 illustrates an example of locations on a user's body for placing vibration apparatuses; and

FIG. 12 illustrates a flowchart of one embodiment of a finger prick procedure utilizing a vibration pain deterrent process.

### DETAILED DESCRIPTION

Referring now to the drawings, wherein like reference numbers are used herein to designate like elements throughout, various views and embodiments are illustrated and described, and other possible embodiments are described. The figures are not necessarily drawn to scale, and in some instances the drawings have been exaggerated and/or simplified in places for illustrative purposes only. One of ordinary skill in the art will appreciate the many possible applications and variations based on the following examples of possible embodiments.

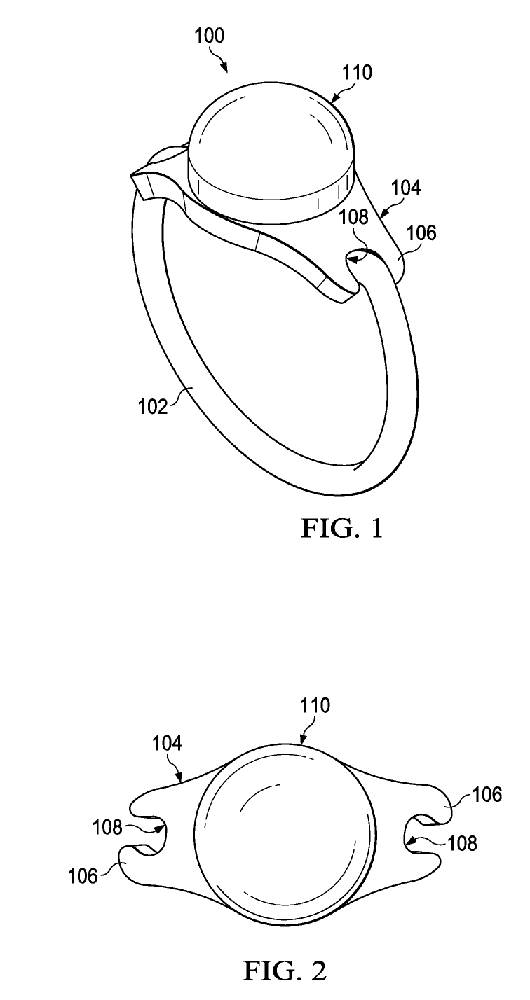

Referring now to FIGS. 1 and 2, there is illustrated one embodiment of a vibrating finger ring 100. The ring 100 includes a circular band 102. The circular band 102 is configured to allow for a finger to be inserted therein. The circular band may be made of plastic, a flexible material such as rubbery, or any other suitable material. Affixed atop the circular band 102 is a securing member 104 having opposing brackets 106. The opposing brackets 106 hold the circular band 102 in place below the securing member 104 by allowing for the circular band 102 to sit securely within openings 108. The opposing brackets 106 thus allow the circular band 102 to be forced and compressed through a narrow entryway and into a larger region of the opening as shown. Fixed atop the securing member 104 is a housing 110 that houses electrical and mechanical components that allow for a vibrating effect, as described herein. The vibrating effect provides an anesthetic effect to the wearer of the ring 100 to assist the wearer in performing a finger prick. The housing 110 and the securing member 104 may twist apart to allow for battery replacements and then twist or snap back together.

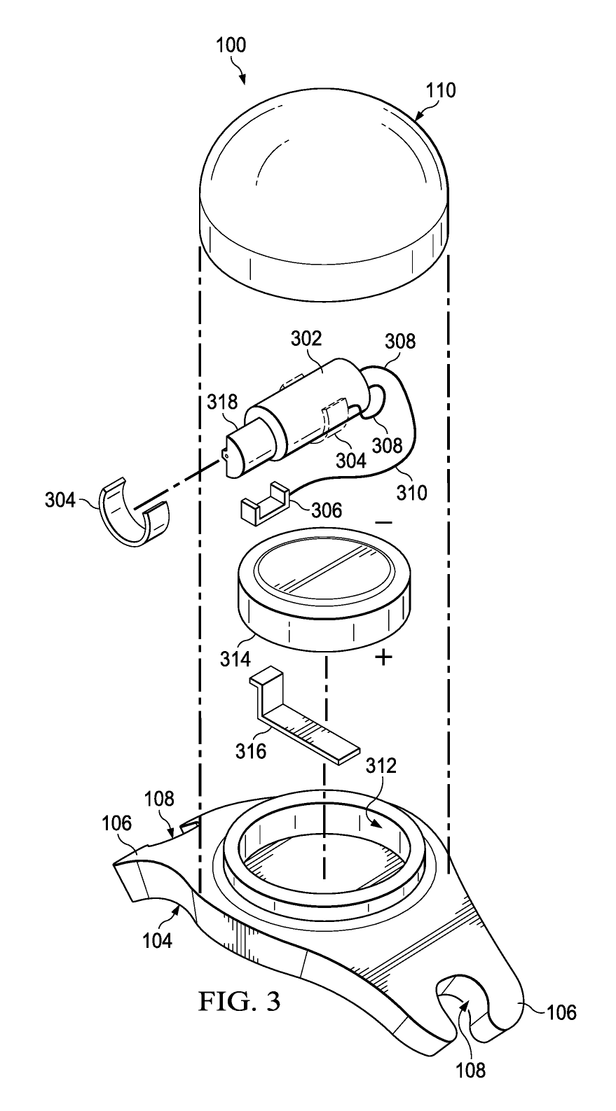

Referring now to FIG. 3, there is illustrated an exploded perspective view of the housing 110 and the securing member 104 showing the internal components that provide the vibrating effect. The housing 110 contains a transversely mounted vibrator motor 302, a negative battery contact 304, and a switch contact 306. The position for the negative battery contact 304 is shown in dashed lines. A negative motor wire 308 is connected to the negative battery contact 304, and a positive motor wire 310 is connected to the switch contact 306. A lower battery housing 312 located within the securing member 104 contains a battery 314 and a positive battery contact 316. The positive battery contact 316 extends upward from the battery housing 312 so that it may touch the switch contact 306 when the unit is assembled. Rotating the housing 110 in relation to the securing member 104 may power the apparatus on and off. When assembled, the battery 314 may be mounted alongside the motor 302. The negative contact 304 may also come into direct contact with the battery 314 when the device is assembled.

The vibrating motor 302 may be a cylindrical DC motor that causes an asymmetrical head 318 to spin to cause the vibrating effect. The motor is preferably 10 mm in length and about 6 mm in diameter or smaller. The motor may also be a flat, pancake, shape of other known vibrating motor, if desired, and oriented similar to the battery 314. For example, the motor could be one such as that manufactured by Jinlong Machinery & Electronics Co., Ltd. of Yeuquing, Zhejiang, China, such as model number Z4KC1B1051202. Rotating the housing 110 in relation to the securing member 104 may operate an on-off switch and, if further rotated, may twist open the housing 110 from the securing member 104 to provide access to the battery. An alternative on-off switch type may also be implemented if desired.

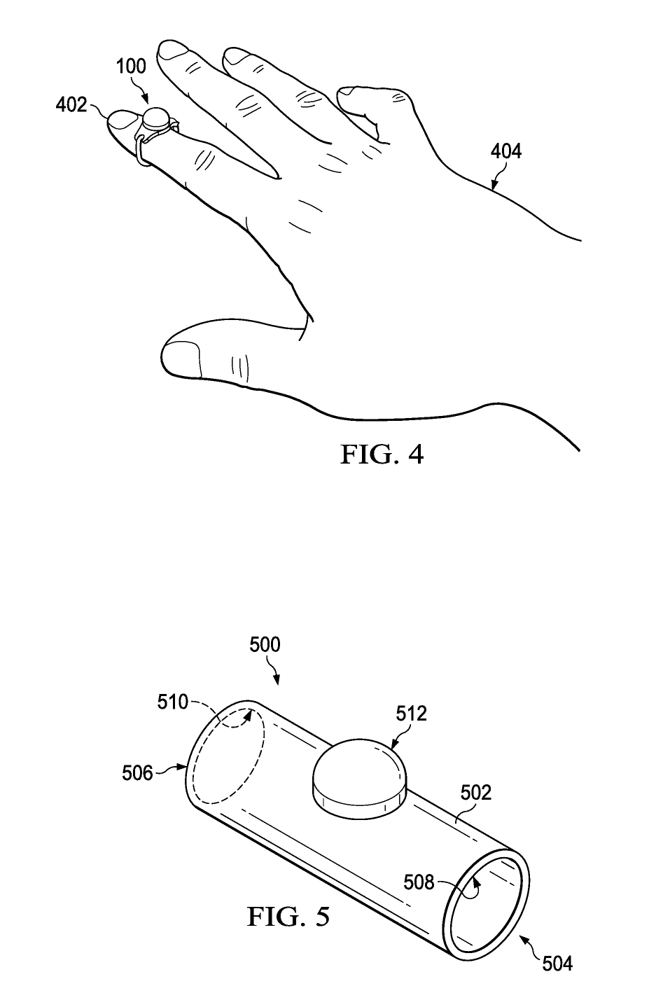

Referring now to FIG. 4, there is illustrated a perspective view showing the ring 100 worn on an index finger 402 of a hand 404. The ring 100 is worn near the fingertip of the index finger 402. It will be understood that the ring 100 may be positioned even closer to the fingertip than that shown in FIG. 4 so as to maximize the vibrating sensation at the end of the index finger 402. Placing the ring 100 near the fingertip of the index finger 402 allows for an anesthetic effect to take place while a finger prick is performed on the index finger 402. It will be understood that rings of different sizes may be provided depending on the size of the hand or when different fingers other than the index finger are intended to be pricked. In embodiments where the circular band 102 is made from a flexible material, a single ring 100 may be used on any finger, with the circular band 102 expanding to fit tightly over any finger on the hand.

Referring now to FIG. 5, there is illustrated one embodiment of a vibrating finger cuff or sleeve 500. The sleeve may be a unitary structure formed from a single piece of plastic, fabric, an elastic material, or other suitable materials. The vibrating finger sleeve 500 includes a hollow body 502 having a first end 504 and a second end 506. The body 502 tapers as it progresses from the first end 504 to the second end 506. A finger is initially inserted through an first opening 508 at the first end 504, traveling through the hollow body 502, and exiting the body 502 via a second opening 510 at the second end 506. Preferably, only a small portion of the finger that includes the fingertip exits the body 502 at the second opening 510. Mounted atop the vibrating finger sleeve 500 is a housing 512. The housing 512 contains electrical and mechanical components that allow for a vibrating effect to take place similar to the housing 110 described herein. A battery may be within a second housing positioned below the housing 512 similar to the securing member 104 described herein, the batter may be additionally included within the housing 512, or the battery may reside in within a cutout of the vibrating finger sleeve 500, with the housing 512 serving to secure and enclose the battery within.

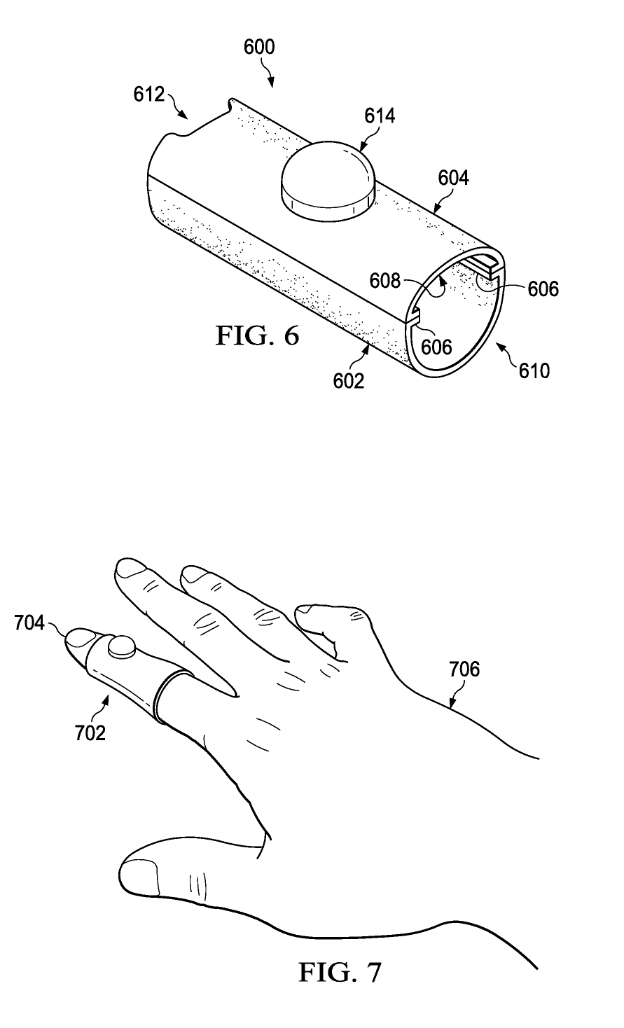

Referring now to FIG. 6, there is illustrated another embodiment of a vibrating finger sleeve 600. The vibrating finger sleeve 600 includes a bottom body portion 602 where the bottom surface of a finger may rest when inserted into the vibrating finger sleeve 600. A top body portion 604 having flexible tabs 606 extending towards the center of and within a cavity 608 is also included. In some embodiments, the top and bottom body portions 602 and 604 may be made from a material that is comfortable to the wearer, such as a soft fabric. In other embodiments, a flexible material such as rubber may be used wherein the sleeve is stretched over the finger so as to provide a tight grip around the finger to increase the vibrating sensation.

The top surface of the finger rests against the top inside surface of the top body portion 604. The flexible tabs 606 may wrap underneath the finger to provide for increased grip on the finger. The finger is inserted into the cavity 608 at a first end 610 of the vibrating finger sleeve 600. The finger is inserted through the cavity 608 until the finger exits the vibrating finger sleeve 600 at a second end 612. Preferably, only a small portion of the fingertip will exit the vibrating finger sleeve 600. Mounted atop the vibrating finger sleeve 600 is a housing 614. The housing 614 contains electrical and mechanical components that allow for a vibrating effect to take place similar to the housing 110 described herein. A battery may be within a second housing positioned below the housing 614 similar to the securing member 104 described herein, the batter may be additionally included within the housing 614, or the battery may reside in within a cutout of the vibrating finger sleeve 600, with the housing 614 serving to secure and enclose the battery within.

Referring now to FIG. 7, there is illustrated a perspective view showing a vibrating finger sleeve 702 worn on an index finger 704 of a hand 706. The sleeve 702 may be similar to the embodiments described with respect to FIGS. 5 and 6. The index finger 704 is inserted into the sleeve 702 until the fingertip of the index finger 402 protrudes from the sleeve 702. It will be understood that the sleeve 702 may be positioned even closer to the fingertip than that shown in FIG. 7 so as to maximize the vibrating sensation at the end of the index finger 704. Placing the sleeve 702 near the fingertip of the index finger 704 allows for an anesthetic effect to take place while a finger prick is performed on the index finger 704. It will be understood that sleeves of different sizes may be provided depending on the size of the hand or when different fingers other than the index finger are intended to be pricked. In embodiments where the sleeve 702 is made from a flexible material, a single sleeve may be used on any one finger, with the sleeve 702 expanding to fit tightly over any finger on the hand.

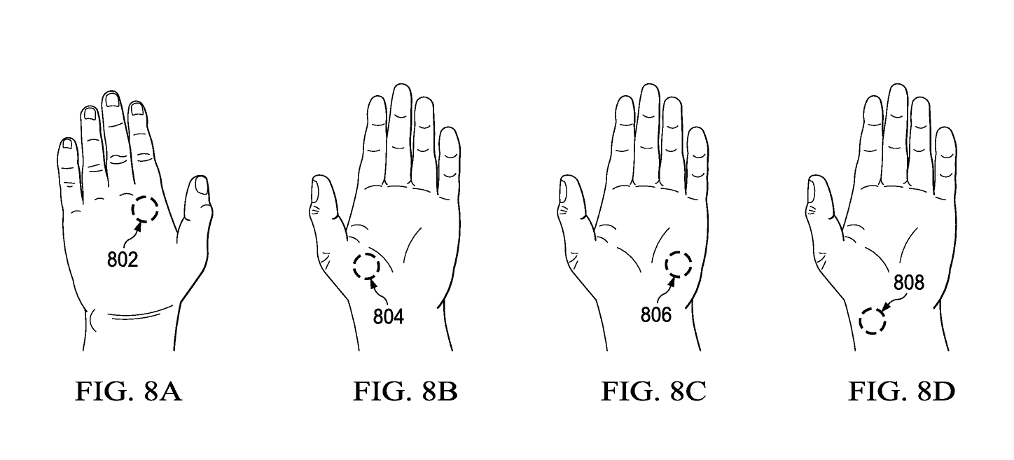

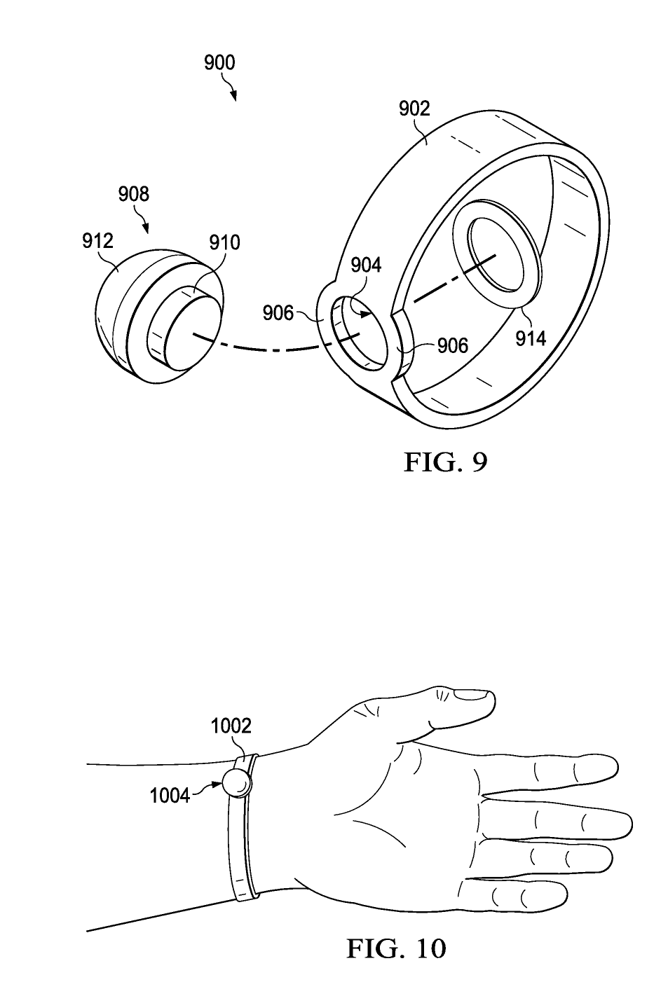

Referring now to FIGS. 8A-8D, there are illustrated various locations on the hand or wrist that may be used with the vibration apparatuses and methods described herein. FIG. 8A shows a vibration location 802 at the dorsum of the hand just proximal to the second knuckle. FIG. 8B shows a vibration location 804 at the thenar eminence. FIG. 8C shows a vibration location 806 at the hypothenar region. FIG. 8D shows a vibration location 808 at the volar wrist.

Vibration applied to these points can affect the tactile sensation of the fingertip pads by applying a vibration effect. Lower vibrations, especially those that are almost imperceptible to the user, may not be effective and may even increase tactile sensation in the fingertip pads. However, applying a stronger vibration effect at these points can decrease tactile sensation in the fingertip pads. Additionally, a vibration effect may be used at any of these points, or a combination of these points, as well as at the fingertip, to even further decrease tactile sensation. For example, a vibrating apparatus may be placed at the thenar eminence, and another vibrating apparatus such as the ring disclosed herein may be placed at or near the fingertip, to even further reduce tactile sensation.

Referring now to FIG. 9, there is illustrated one embodiment of a vibrating band 900. The band 900 may be a circular or arcuate shape having a flat circular body 902 of a particular width. In some embodiments, the width of the flat circular body 902 may be minimal so as to reduce bulkiness for the wearer/user of the band 900. The circular body 902 may be made of a flexible material so that the band can be stretched over a user's hand, wrist, or other location. In other embodiments, the circular body 902 may be made of a metallic material that is sized for the desired vibration location. Still other materials may be used, such as plastic, or others. The flat circular body 902 may have an arcuate aperture 904 situated at one location on the band 900. In the embodiment shown in FIG. 9, curved surfaces 906 curve around the arcuate aperture 904 and connect between two points of the flat circular body 902. The arcuate aperture 904 allows for a housing 908 containing a vibration mechanism, such as one similar to that shown in FIG. 3, to be slotted into the arcuate aperture 904.

The housing 908 may have a cylindrical body 910 disposed underneath a circular head portion 912, so that the cylindrical body 910 is inserted into the arcuate aperture 904, while the circular head portion 912 prevents the entire housing from passing through the arcuate aperture 904. A fixing member 914 may then be applied to the cylindrical body 910 to hold the housing 908 in place within the arcuate aperture 904 and on the band 900. For example, the fixing member 914 may be a ring as shown in FIG. 9 that is of the proper size to be inserted over the cylindrical body 910 and compress against the cylindrical body 910 to hold the housing in place. Other types of fixing members may also be used, such as clasps, hooks, adhesive, screws, or others. Once the housing 908 is fixed onto the band 900, the band 900 may be placed on the desired location, with the housing 908 placed over the desired vibration location, such as locations 802, 804, 806, and 808, as shown in FIGS. 8A-8D, to apply vibration to that chosen location.

Referring now to FIG. 10, there is illustrated one example of a vibration location placement. There is shown a vibrating band 1002 having a vibration mechanism in a housing 1004. This band may be similar to that described with respect to FIG. 9. The band 1002 is shown placed around the wrist of a user, with the housing 1004 containing the vibration mechanism placed over the volar wrist. Vibration may be applied to this area to decrease sensitivity in the fingertips of the user. The band 1002 may be placed over any location that may allow for vibration to decrease fingertip sensitivity. For example, the band may be inserted over a user's hand just over the knuckles to allow the housing 1004 to reach the dorsum of the hand just proximal to the second knuckle (location 802), or may be placed at other locations such as locations 804, 806, or other desired locations that may be proven effective.

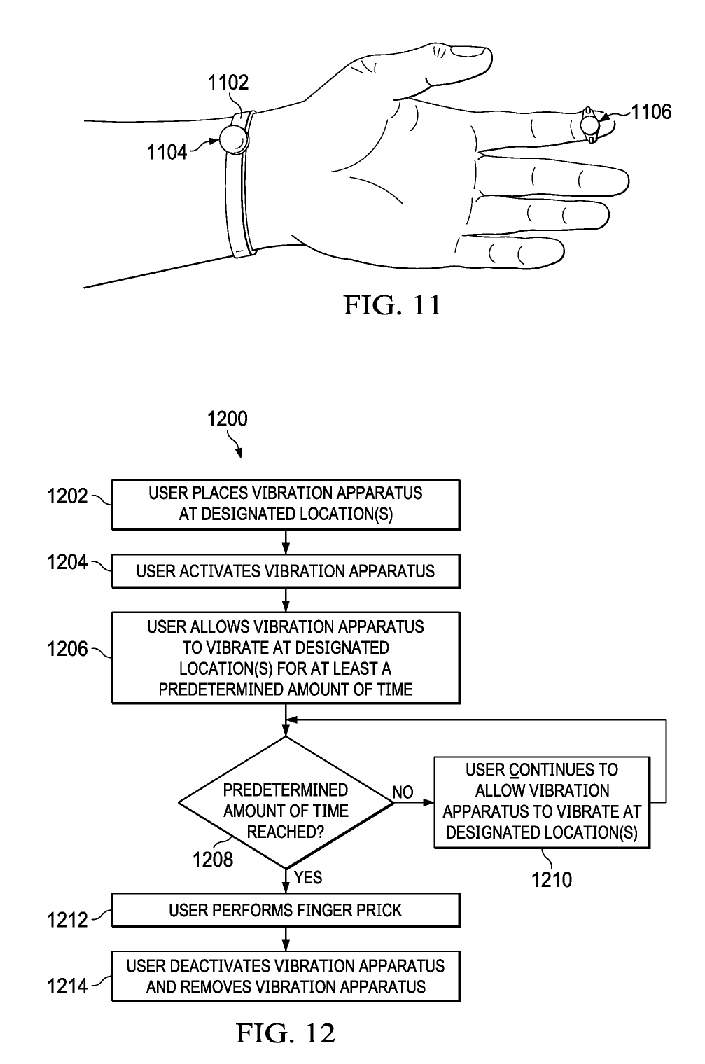

Referring now to FIG. 11, there is illustrated another example of a vibration location placement. There is shown a vibrating band 1102 having a vibration mechanism in a housing 1104. This band may be similar to that described with respect to FIG. 9. The band 1102 is shown placed around the wrist of a user, with the housing 1104 containing the vibration mechanism placed over the volar wrist. Vibration may be applied to this area to decrease sensitivity in the fingertips of the user. The band 1102 may be placed over any location that may allow for vibration to decrease fingertip sensitivity. For example, the band may be inserted over a user's hand just over the knuckles to allow the housing 1104 to reach the dorsum of the hand just proximal to the second knuckle (location 802), or may be placed at other locations such as locations 804, 806, or other desired locations that may be proven effective. The example shown in FIG. 11 further includes another vibration apparatus 1106, such as a vibration ring as described herein, placed at or near the finger tip of the finger which is to be subject to a finger prick or other painful operation. The extra placement of a vibration apparatus at the fingertip can further decrease sensitivity in the fingertip when coupled with the placement at the volar wrist or other locations.

Referring now to FIG. 12, there is illustrated a flowchart of one embodiment of a finger prick procedure utilizing a vibration pain deterrent process 1200. The process 1200 begins at step 1202 where a user places a vibration apparatus at a designation location on the user's body. The vibration apparatus may be any of the vibration apparatuses described herein, such as a finger ring, sleeve, or band and the designated location on the user's body may be the finger which the user intends to perform the finger prick on, a location on the hand or wrist that is effective in decreasing fingertip sensitivity when a vibration effect is applied (such as locations 802, 804, 806, and 808), other locations that are effective in decreasing fingertip sensitivity, or any combination of these locations. If more than one location is to be used, a separate vibration apparatus may be placed at each location, with the type of vibration apparatus being different depending on the location.

The process 1200 then flows to step 1204, where the user activates the vibration apparatus, or multiple vibration apparatuses if multiple locations are designated. At step 1206, the user allows the vibration apparatus or apparatuses to vibrate at the designated location(s) for at least a predetermined amount of time. The predetermined amount of time is an amount of time necessary for the vibration effect to cause decreased sensitivity in the fingertip that is to be pricked. This amount of time may be enough for decreased sensitivity to begin, but not too long so as to allow the user to become desensitized to the vibration sensation. For example, the predetermined amount of time may be 20 seconds from when the user activates the vibration apparatus or apparatuses in step 1204. However a full minute might be too long, and so the user may be urged to perform the finger prick shortly after 20 seconds from activation.

The process 1200 then flows to decision block 1208, where it is determined if the predetermined amount of time has been reached. If not, the process 1200 flows to step 1210 where the user continues to allow the vibration apparatus or apparatuses to vibrated at the designated location(s). From step 1210, the process then flows back again to decision block 1208. If at decision block 1208 it is determined that the predetermined amount of time has been reached, the process flows to step 1212. At step 1212, the user performs finger prick on the finger affected by the vibration apparatus or apparatuses. At step 1214, after the finger prick is fully performed at step 1212, the user deactivates the vibration apparatus or apparatuses and removes the vibration apparatus or apparatuses.

It should be understood that the drawings and detailed description herein are to be regarded in an illustrative rather than a restrictive manner, and are not intended to be limiting to the particular forms and examples disclosed. On the contrary, included are any further modifications, changes, rearrangements, substitutions, alternatives, design choices, and embodiments apparent to those of ordinary skill in the art, without departing from the spirit and scope hereof, as defined by the following claims. Thus, it is intended that the following claims be interpreted to embrace all such further modifications, changes, rearrangements, substitutions, alternatives, design choices, and embodiments.

1. A vibration apparatus for use in performing a finger prick, comprising:
a hollow body;
a housing secured to an outside surface of the hollow body, the housing including:
a vibrator motor; and
a battery housing including a battery.

2. A method for performing a finger prick using a vibration apparatus, comprising:
placing the vibration apparatus at a designated location on a user's body;
activating the vibration apparatus to initiate a vibration effect at the designated location;
allowing the vibration effect to continue at the designated location for at least a predetermined amount of time; and
performing a finger prick at one of a user's fingertips upon reaching at least the predetermined amount of time.

3. The vibration apparatus of claim 1, wherein the housing further includes:
a negative battery contact;
a switch contact;
a negative motor wire connected between the vibrator motor and the negative battery contact; and
a positive motor wire connected between the vibrator motor and the switch contact.

4. The vibration apparatus of claim 3, wherein the battery housing further includes a positive battery contact.

5. The vibration apparatus of claim 4, wherein the positive battery contact extends upward from the battery housing so that it contacts the switch contact, and wherein the negative battery contact contacts the battery.

6. The vibration apparatus of claim 1, wherein the hollow body includes a first end and a second end, wherein the first end includes a first opening and the second end includes a second opening.

7. The vibration apparatus of claim 6, wherein a finger is inserted into the first opening until the finger exits the hollow body at the second opening.

8. The vibration apparatus of claim 6, wherein a hand is inserted into the first opening until and through the second opening such that the hollow body comes into contact with a wrist.

9. The method of claim 2, wherein the vibration apparatus includes:
a hollow body that can be placed around the designated location; and
a housing fixedly coupled to the hollow body, the housing including a vibration motor and a battery.

10. The method of claim 9, wherein the hollow body includes a first end and a second end, wherein the first end includes a first opening and the second end includes a second opening.

11. The method of claim 10, wherein a finger is inserted into the first opening until the finger exits the hollow body at the second opening.

12. The method of claim 10, wherein a hand is inserted into the first opening until and through the second opening such that the hollow body comes into contact with a wrist.

13. The method of claim 9, wherein the housing further includes:
a negative battery contact;
a switch contact;
a negative motor wire connected between the vibrator motor and the negative battery contact; and
a positive motor wire connected between the vibrator motor and the switch contact.

14. The method of claim 13, wherein the battery housing further includes a positive battery contact.

15. The method of claim 14, wherein the positive battery contact extends upward from the battery housing so that it contacts the switch contact, and wherein the negative battery contact contacts the battery.

16. The method of claim 2, further comprising:
deactivating the vibration apparatus; and
removing the vibration apparatus from the designated location.
